(19) United States
(12) Plant Patent Application Publication
(10) Pub. No.: US 2003/0074702 P1
(43) Pub. Date: Apr. 17, 2003

(54) Hybrid tea rose plant named 'Olijplam'

(76) Inventor: Huibert W. Olij, De Kwakel (NL)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/301,741

(22) Filed: Nov. 22, 2002

Related U.S. Application Data
(63) Continuation of application No. 09/382,592, filed on Aug. 25, 1999.

(51) Int. Cl. A01H 5/00

(52) U.S. Cl. PLT/140

(57) ABSTRACT

A new and distinct variety of Hybrid Tea rose plant is provided which forms attractive double dark red blossoms. The plant exhibits an erect growth habit thick sterns, and dark green and very glossy roliage. Excellent disease resistance is exhibited. The new variety is particularly well suited for producing cut flowers under greenhouse growing conditions.

### SUMMARY OF THE INVENTION

The new variety of Hybrid Tea rose plant was created by arificial pollination wherein two parents were crossed which previously had been studied in the hope that they would contribute the desired characteristics. The female parent (i.e., the seed parent) of the new variety was the ‘Pekcoujenny’ variety (United States Plant Pat. No 7,749). The male parent (i.e., the pollen parent) was the ‘Ridriko’ variety (U.S. Plant Pat. No. 7,362). The parentage of the new variety can be summarized as follows:

‘Pekcoujenny’×‘Ruidriko’.

The seeds resulting from the above pollination were sown and small plants were obtained which were physically and biologically different from each other. Selective study resulted in the identification of a single plant of the new variety.

It was found that the new variety of Hybrid Tea rose plant of the present invention possesses the following combination of characteristics:

(a) forms attractive double dark red blossoms,

(b) exhibits an erect growth habit,

(c) forms thick stems,

(d) forms dark green and very glossy foliage, and

(e) is particularly well suited for the production of cut flowers under greenhouse growing conditions

The dark red blossoms contrast nicely with the very Mossy drk grew foliage.

The new variety well meets the needs of the horticultual industry an exhibits good productivity during cut flower production in a greenhouse

The new variety has been found to undergo asexual propaganon in France by a number of routes, including budding, grafting, and cutta. Aseiual propagation by the above-mentioned techniques in France has shown that the characteristics of the new variety are stable and are strictly transmissible by such asemal propagation from one generation to another.

The new variety has been named the ‘Olijplam’ varicy.

### BRIEF DESCRIPTION OF THE PHOTOGRAPH

The accompanying photograph shows as nearly true s it is rasoniaby possible to make the same, in a color illustration of this character, typical specimens of the plant parts of the new variety. The rose plant of the new variety were two years of age and were observed during November while budded on Rosa indica understock and growing in greenhouses at Le Canner des Mmres, Var, France. Dimensions in centimeters are indicated at the bottom of the photograph.

FIG. 1—ilustrates a specimen of a young shoot;

FIG. 2—illstrates a specimnen of a floral bud before the openig of the sepals;

FIG. 3—illustrmtes a specimen of a floral bud at the opening of the seals;

FIG. 4—illustrates a specimen of a floral bud at the openinog of the petals;

FIG. 5—illustrates a stecirnen of a flower in the course of openiag;

FIG. 6—illustrates a specirnen of an open flower—plan view obverse;

FIG. 7—illustrates a stecimen of an open flower—plan view revere;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface; and

FIG. 15—illustrates a specimen of a leaf with five leaflets—plan view—under surface.

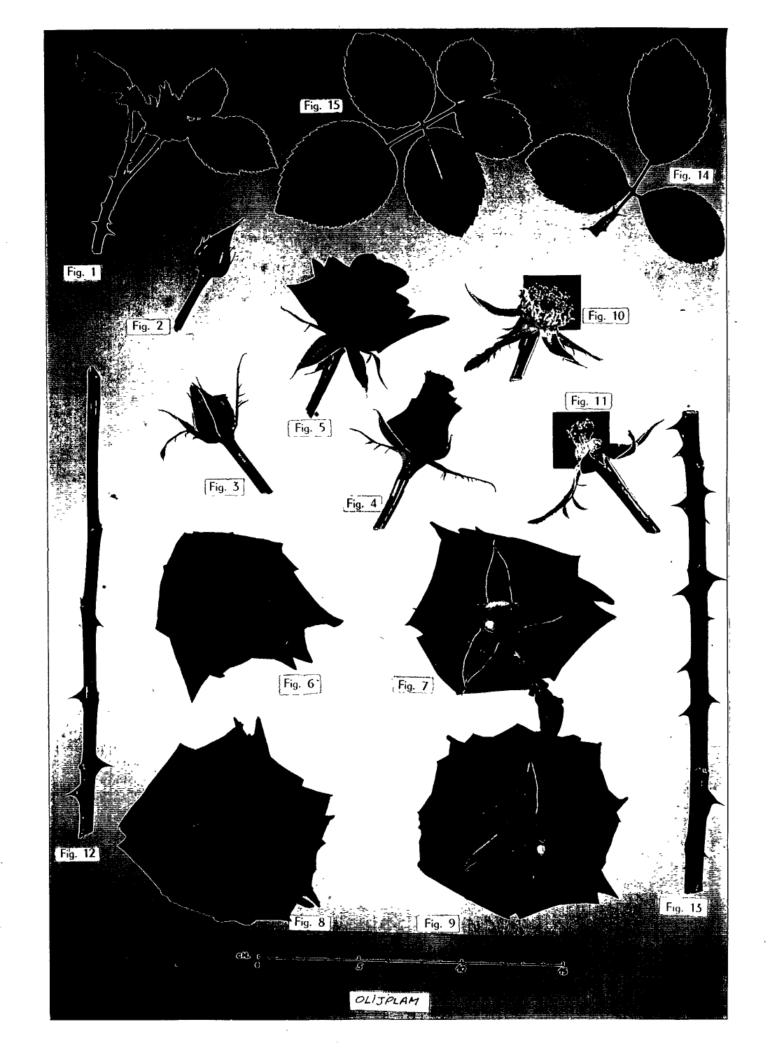

### DETAILED DESCRIPTION

The chart used in the ideinfi cation of the colors is that of the Royal Horticultural Society (R.H.S. Colour Chart). The description is based on the observation of three year-old plants during April while budded on Rosa indica understock and growing indoors at Le Cannet des Maures, Var, France. The coloration in common tarms precedes reference to the chart in some instances.

CLASS: Hybrid Tea.

PLANT: heigh—when pruned to a height of 85 cm., floral stems of approximately 60 to 80 cm. in length commonly are produced.

habit.—erect.

BRANCHES: color—young stems: Yellow-Green Group 147 B suffused with reddish brown. adult wood: Yellow-Green Group 147 B sufflised with reddish brown.

thorns—size: large (as illustrated). qantity: numerous (as illustrated). color: Frown to reddish. configuration: concave on the under surface

LEAVES: stipules—adnate. pectinate, broad and linear.

petoles—upper surface: striped reddish brown on young foliage and dark green on adult foliage. under surface: light green and smooth.

leadets—number: 3, 5 (most often), and 7. shape: rounded (as illustrated). serraticn: single and regular (as illustrated) texture: consistent. general appearance: dense, dark green, and very glossy. color (young foliage): upper surface: dark green, near Yellow-Green Group 147 A and sufflused with reddish brown. under surface: dark green, Yellow-Green Group 147 A and suffused with reddish brown. color (adult foliage): uppersurface: dark green near Yellow-Green Group 147 A. under surface: dark green, near Yellow-Green Group 147 B.

### INFLORESCENCE

number of flours—commonly 1 to 3 per stem.

peduncle.—medium to dark green, with few prickles, and the length is approximately 10 cm. on average.

sepals.—upper surface: tomentose and greenish in coloration. under surface: medium green in coloration and commonly with a few extensions (as illustrated)

buds.—conical and elongated. size: medium. lenth: approximately 5.5 cm. on average. color upon opening: upper surface: near Cardinal Red, Red Group 53 A, with a velvety aspect. under surface: near Cardinal Red, Red Group 53 A and tinted with Greyed-Purple Group 185 A.

flower.—shape: cup-shaped with parallel sides. diameter: commonly approximately 13 cm. on average. color (when orening begins): upper surface: commonly between Red Group 46 A and Red Group 46 B. under surface: near Red Group 46 A and lightly suffused with Red-Purple Group 59 A. color (when blooming): upper surface: Red Group 46 A under surface: near Red Group 46 A color (a end of opening): upper surface: rear Red Group 45 B under race: te exterml petals commonly are near Red Group 46 A, and the interal petals commolnly are near Red Group 46 A and are suffesed with Red Group 53 B fragrance: none lasting quality: approximately 9 to 10 days on the plant and approximately 7 to 8 days when cut and placed in a vase petal number: commonly approximately 45 on average petal shape: rounded with reflexed edges. petal drop: good, the peals commonly detach cleanly. staman number approximately 250 on average. anothers: golden yellow in coloration. filaments: reddish in coloration. pistils: approximately 160 on average. styles: yellowish-pink in coloration. styles: yellowish-pink in coloration. receptacle: medium green im coloration, smooth, and in longitudinal section in the shape of a funnel. hips: these are formed.

### DEVELOPMENT

vegetation.—strong.

blooming.—abundant with approximately 140 to 170 blossoms per square meter per year being formed under greenhouse growing conditions.

resistance to diseases.—very good.

I clam:

1. A new and distinct variety of Hybrid Tea rose plant charaterized by the following combination of characteristics:
(a) forms attractive double dark red blossoms,
(b) exhibits an erect growth habit,
(c) forms thick stems,
(d) forms dark green and very glossy foliage, and
(e) is particularly well suited for the production of cut flower under greenhouse growing conditions; substantially as herein shown and described.
